(19) United States
(12) Patent Application Publication
(10) Pub. No.: US 2010/0172845 A1
(43) Pub. Date: Jul. 8, 2010

(54) COMPOSITIONS AND METHODS FOR TREATING MYCOBACTERIAL INFECTIONS

(75) Inventors: James K. Stoops, Houston, TX (US); Robert L. Hunter, Bellaire, TX (US); Semyon A. Risin, Houston, TX (US)

Correspondence Address: HAYNES AND BOONE, LLP;IP Section, 2323 Victory Avenue, Suite 700, Dallas, TX, 75219, US

(73) Assignee: BOARD OF REGENTS OF THE UNIVERSITY OF TEXAS SYSTEM, Austin, TX (US)

(21) Appl. No.: 12/601,494

(22) PCT Filed: May 29, 2008
(86) PCT No.: PCT/US08/65127

§ 371 Date: Feb. 22, 2010

Related U.S. Application Data
(60) Provisional application No. 60/940,577, filed on May 29, 2007.

(51) Int. Cl. A61K 9/12; A61K 47/00; A61P 31/04

(52) U.S. Cl. 424/45; 514/777

(57) ABSTRACT

Disclosed are compositions and improved methods for effective treatment of Mycobacterial infections in susceptible animals. Also disclosed are regimens for preventing, reducing, or ameliorating the emergence of symptoms of Mycobacterium tuberculosis infection in susceptible individuals, as well as methods for reducing the spread of tubercular infections in at-risk populations.

### CROSS-REFERENCE TO RELATED APPLICATIONS

The present application claims priority to U.S. Provisional Application Ser. No. 60/940,577, filed May 29, 2007 (now abandoned), the entire contents of which is specifically incorporated herein by reference in its entirety.

### STATEMENT OF FEDERALLY-SPONSORED RESEARCH

The United States government may have certain rights in the present invention pursuant to National Heart, Lung, and Blood Institute Grant number 5RO1-HL068537-05 from the National Institutes of Health.

### FIELD OF THE INVENTION

The present invention relates generally to the fields of medicine, and more specifically to the treatment of Gram-positive bacterial infections. In particular, compositions and methods are disclosed for the treatment, amelioration of symptoms, and prophylaxis of pathogenic Mycobacterial infections, including, for example, infection by Mycobacterium tuberculosis.

### DESCRIPTION OF RELATED ART

### Mycobacteriaceae

The Mycobacteriaceae are a family of aerobic, acid-fast, non-motile, Gram-positive bacteria of the order Actinobacteria. The family comprises several species that are highly pathogenic in mammals, including the known causative agents of tuberculosis (“TB”) (Mycobacterium tuberculosis; “MTB”), leprosis (also known as “leprosy” or “Hansen's disease”) (M. leprae), and bacterial sepsis in immunocompromised and/or HIV-infected individuals (M. avium complex; “MAC”). Also within the genus are a number of other species that are pathogenic to one or more mammalian species, including, for example, Mycobacterium africanum, Mycobacterium avium, Mycobacterium avium paratuberculosis, Mycobacterium avium silvaticum, Mycobacterium avium hominissuis, Mycobacterium avium colombiense, Mycobacterium botniense, Mycobacterium bovis, Mycobacterium canetti, Mycobacterium caprae, Mycobacterium haemophilum, Mycobacterium heckeshornense, Mycobacterium intracellulare, Mycobacterium kansasii, Mycobacterium microti, Mycobacterium paratuberculosis, Mycobacterium pinnipdeii, Mycobacterium ulcerans and Mycobacterium xenopi.

### Mycobacterium Tuberculosis and Mycobacterium Leprae

According to World Health Organization (“WHO”) estimates, nearly two billion people (one-third of the world's population) are thought to be infected with M. tuberculosis, while three to five million are permanently disabled due to M. leprae infection. While the majority of those infected with do not show clinical signs of disease, each year about nine million people develop active TB, and nearly two million die from it (Dye et al., 1999). TB is the leading cause of death from a single bacterial infection, and the leading cause of mortality in persons infected with HIV.

Despite aggressive treatment protocols in use for more than a century, M. tuberculosis infection remains one of the most serious threats to world health, particularly in developing and third-world nations. A key reason that the pathogen remains a significant health risk for the human population relates to its remarkable ability to persist for long periods of time in the host body, even in the face of immunity, chemotherapy, and/or antimicrobial therapy. The emergence of MDR strains of the bacterium has not only exacerbated successful treatment, but also increased the arsenal of potential bioterrorism agents.

Studies have shown that M. tuberculosis infection in humans can persist for decades (World Health Organization, 1989; Russell, 2001). Moreover, the development of MDR strains of the bacterium, the epidemic spread of HIV infections among compromised individuals, and the reactivation of latent TB in the general population all increase the risk factors for disease development and account for nearly one-third of AIDS-related deaths in HIV infected individuals.

In vitro, the Mycobacteria are highly fastidious (i.e., difficult to culture) sometimes taking over two years to develop in culture. Many species also have extremely long reproductive cycles that make laboratory culture and identification of the organism a slow process. M. leprae, for example, can take three weeks to proceed through one division cycle; in comparison, strains of E. coli often take 20 min or less.

MTB pathogenicity is related to its ability to escape destruction by macrophages and other components of the immune system to induce delayed-type hypersensitivity (Saita et al., 1997; Ellner, 1977). This phenomenon has been attributed to several components of the M. tuberculosis cell wall, and in particular, a surface glycolipid known as “cord factor” (trehalose 6,6′-dimycolate; “TDM”). It has been shown that virulent strains of M. tuberculosis express cord factor on their surfaces, while non-virulent strains of the bacterium do not

TDM surrounds the bacteria with a unique sticky surface that firmly holds them together to ultimately form structures named “cords.” It is this superstructure which forms a hard, impenetrable shield that covers the aggregated bacteria and protects them from macrophages, neutrophils and other components of the immune system. These cords may result in necrosis of the surrounding lung tissue to give rise to a cavity (“cavitation”) that is associated with clinical symptoms of the disease. Even though individual bacteria may be found throughout the lung, they fail to form cords because lung surfactant retards their aggregation.

This brings into consideration the significance of the bacterium's lipid-rich bilayer surface. Cholesterol enters the outer bilayer and reduces its fluidity, rendering it harder to the extent that the viability of bacteria may be compromised. Space-filling models of TDM's structure often fail to represent the kinks in the fatty acid chain introduced by the cyclopropane rings interspersed throughout the chain. This component introduces notable kinks in the chain much like the cis double bond, and both moieties have been demonstrated to increase the fluidity of the membrane by the introduction of gaps between the chains. Model systems of lipid bilayers and in vivo bacterial models have demonstrated that cholesterol may enter and fill these gaps, and consequently reduce the fluidity of the membrane.

In human beings, M. tuberculosis grows in the endobronchial space and occasionally in the alveoli of infected individuals, where it results in the inflammation and progressive destruction of the lungs. Other manifestations of the disease include fever and a recurrent, nonproductive cough.

### Symptoms of Mycobacterium Tuberculosis Infection

In industrialized societies, tubercular infections tend to be concentrated among inner city dwellers, ethnic minorities, and recent immigrants from areas of the world where the disease is endemic. Alcoholics (who are often malnourished) are high risk for developing the disease, as are immunocomprised individuals, including those infected with human immunodeficiency virus (“HIV”).

M. tuberculosis, the causative agent of TB is transmitted from one individual to another by inhalation of mycobacterial-infested airborne droplets, typically among persons in very close contact. Only about 10% of those initially infected with M. tuberculosis, however, will actually develop the active form of the disease. The first symptoms of active case often present themselves as a “cold” or influenza. For people with the disease, TB can cause lung or pleural disease or it may spread through the body via the circulatory system. Infected individuals often do not seek medical attention until they have pronounced symptoms, such as pleurisy or hemoptysis (i.e., the coughing up of blood). Other symptoms often include fever, loss of appetite, weight loss and/or nocturnal hydrosis. About 15% of individuals presenting with an active TB infection will develop the disease in an organ other than the lung, including for example, the lymph nodes, the gastrointestinal tract, bone and joints.

### Anti-Tubercular and Anti-Mycobacterial Agents

Despite significant advances in antimicrobial therapies over the past four decades, contemporary treatment regimens for the eradication of TB and other pathogenic Mycobacteria are largely ineffective. The WHO's current treatment guidelines for TB-infected individuals require a minimum six- to nine-month multidrug treatment regimen that includes a combination of at least four of the so-called “first-line” antibiotics: isoniazid (“INH”), rifampin (“RFP”), rifabutin (“RFB”), rifapentine (“RPT”), pyrazinamide (“PZA”) and ethambutol (“EMB”). Unfortunately, these lengthy treatments are both expensive and suffer from poor patient compliance, a problem exacerbated by the toxic side effects that often occur during prolonged treatment regimens.

Moreover, the addition of one or more “second-line” (such as cycloserine, ethionamide, moxifloxacin, ofloxacin, levofloxacin, capreomycin, streptomycin, kanamycin and p-aminosalicylic acid [“PSA”], etc.), or “third-line” antibiotics (such as clofazimine, imipenem, linezolid, amoxicillin, etc.) often provide little additional treatment benefit, unless the prescribed drug regimen is rigorously followed, and the antibiotic “cocktail” administered for the entire duration of the protocol as set forth in WHO guidelines.

A number of so-called “next-generation” antitubercular drugs are currently in development and/or clinical trials, although none have emerged as a clear solution for eradicating the bacterium in infected populations. Among the compounds currently being developed are the nitroimidazole “PA-824” (6S)-2-nitro-6-{[4-(trifluoromethoxy)benzyl]oxy}6,7-dihydro-5H-imidazo[2,1-b][1,3]oxazine) [Chiron/Novartis], nitroimidazole-oxazoles, including “CGI17341” (2-ethyl-5-nitro-2,3-dihydro[2-1b]imidazo-oxazole and “OPC-87683” [Otsuka], “pyrrole LL-3638” [Lupin, Ltd.], as well as a number of substituted quinolones, long chain enol-acyl carrier protein reductase (InhA) inhibitors [inter alia Ciba-Geigy and Incyte], and a number of pleuromutilins derivatives [inter alia, Sandoz, GSK, and Novartis].

### Treatment Regimens for Mycobacteria

In the United States, existing therapeutic regimens for the treatment of TB have been detailed by the National Institutes of Health (“NIH”) and the United States Department of Health and Human Services's Centers for Disease Control and Prevention (“CDC”) and are generally based on results obtained in a number of clinical trials developed by the United States Public Health Service and the Infectious Diseases Society of America. Their most recent joint report contains current guidelines for management of the disease, and outlines the recommended multi-drug treatment protocols for combating tubercular disease (cf. American Thoracic Society et al., 2003). As noted above, many of these protocols require continuation of therapy for at least 4 to 12 months, a fact that both significantly increases the cost of treatment, and decreases the frequency of patient compliance.

According to the NIH, the spread of TB has reemerged as an urgent health problem, as rates for this disease have been on the increase since the mid-1980s. Studies by the CDC show that the number of new active cases of TB reported annually in the U.S. population is now approximately one in ten thousand and approximately eighty in ten thousand in HIV-infected populations (NIH Guideline #HL-95-012, 1995).

Conventional antitubercular therapies involving rifampin and streptomycin frequently have serious side effects. The active disease must be treated using combinations of three or four drugs over a period of at least six to nine months to insure that disease will not recur after treatment is discontinued and to prevent the emergence of resistant strains.

Multidrug-resistant (“MDR”) TB also represents a new challenge. Recently, MDR TB has been recorded at the highest rates ever, according to the latest WHO report that presents the findings from the largest survey to date on the scale of drug resistance in TB including data collected from 2002-2006 on over 90,000 TB patients in 81 countries worldwide.

### Deficiencies in the Prior Art

For these and other reasons, what are lacking in the prior art are compositions and methods for the effective, inexpensive, and short-term treatment and/or prophylaxis of mycobacterial infections, and particularly short-duration therapy regimens for combating MTB infections in affected or at-risk individuals. Particularly lacking are those methods that afford multiple benefits such as (1) higher patient compliance than conventional therapies; (2) widespread availability of the therapeutic agent; (3) fewer side-effects than conventional therapies; and (4) financial affordability of the drug regimen.

Conventional treatment regimens often prove unsuccessful, largely due to a lack of compliance with the multi-week therapies available today. Moreover, it is considered by many in the medical community that the poor compliance of conventional multi-drug regimens not only augments the natural spread of drug-resistant variants of the bacteria, but also exacerbates the availability of financially-affordable treatments for the disease.

In particular, there is a dire need for the discovery of new drugs useful in the treatment of tubercular infections, and development of treatment protocols that could effectively abrogate the transfer of virulent MTB from diseased persons to new recipients. Moreover, there is a critical need for adequate means of preventing the drug in susceptible individuals, and for reducing the spread of MDR forms of the microorganism. The humanitarian and economic benefits of affordable and effective interventions are tremendous.

### SUMMARY OF THE INVENTION

The present invention overcomes these deficiencies and other limitations inherent in the prior art by providing effective, inexpensive compositions and methods for effective and inexpensive short-term regimens for treating and/or preventing mycobacterial infections (and in particular, M. tuberculosis infections), in affected and/or susceptible animals. The present invention provides compositions that have been shown to reverse the corded state of MTB, ostensibly by reducing and/or removing the TDM coverage that encapsulates the bacterial cells. Consequently, the pathogenic abilities of this mycobacterial species are reduced, and as a result, the cells may become susceptible to clearance by the immune system, or even more effectively by short-term application of one or more anti-mycobacterial agent (e.g., antibiotics, antibacterials, antimicrobials, etc.).

In addition, the compounds disclosed herein, and compositions that comprise, consist essentially of, or consist of them, have been shown to be toxic to mycobacterial species, and can thus be used as either an anti-tubercular monotherapy, or alternatively, in combination with one or more known therapeutic agent(s) to provide cost-effective, short-term regimen(s) that cure or significantly diminish the dynamics of the pulmonary TB process in vivo.

In a first embodiment, the invention provides a method for ameliorating at least one symptom of a mycobacterial infection in a mammal. In an overall and general sense, the method involves administering to a mammal in need thereof, an amount of a composition comprising at least a first surface-active agent (surfactant) compound (i.e., detergent) for a time sufficient to ameliorate one or more symptoms of a mycobacterial infection in the infected animal. In exemplary embodiments, the invention provides a method for ameliorating at least one symptom resulting from M. tuberculosis infection in a mammal in which an antimicrobial amount of a composition comprising at least a first surface-active agent (surfactant) compound (i.e., detergent) is administered in an amount and for a time sufficient to ameliorate one or more symptom of M. tuberculosis infection in the infected animal.

In a second embodiment, the invention provides a method for altering, modifying, or reducing the length, severity, or spread of a mycobacterial infection in a mammal. This method generally involves the administration of an amount of a composition that comprises, consists essentially of or alternatively, consists of at least a first surfactant or detergent compound to a mammal in need thereof, for a time sufficient to alter, modify or reduce the length, severity or spread of a mycobacterial infection in the infected individual.

In a third embodiment, the invention provides a method for treating or eradicating a mycobacterial infection in a mammal. In an overall and general sense, this method involves administering to such a mammal a therapeutically-effective amount of a composition that comprises or consists essentially of at least a first surface-active compound for a time sufficient to treat the symptoms of the mycobacterial infection in the infected animal, or to substantially eradicate the pathogenic organism from the infected mammal.

In a fourth embodiment, the invention provides method for decreasing the likelihood of contracting a mycobacterial infection in a mammal that has been exposed to the bacterium. The method comprises at least the step of administering to such a mammal an amount of a composition that comprises, consists essentially of or, alternatively, consists of at least a first surfactant compound for a time sufficient to decrease the likelihood of contracting the mycobacterial infection upon exposure of the individual to a population of mycobacteria.

In a fifth embodiment, the present invention also provides a method for preventing the development of an active mycobacterial infection in a selected mammal. Such methods are particularly contemplated to be useful in areas where the incidence of mycobacterial infections is substantial, and in areas where the organism may be in endemic or epidemic proportion. The method is also particularly contemplated to be useful in persons who are placed at higher risk for contracting the organism, such as for examples, persons deployed to one or more areas of increased mycobacterial prevelance, or for persons in the practice of providing health-care to one or more infected individuals. This method generally involves administering to such an individual a prophylactically-effective amount of a composition that comprises, consists essentially of, or consists of at least a first surfactant compound in an amount and for a time sufficient to retard, lessen, alter, inhibit, or prevent the development of a mycobacterial infection in such a person, or to lessen the opportunity for acquiring one or more diseases caused by exposure to one or more mycobacterial species.

In a sixth embodiment, the invention provides a means for achieving greater patient compliance in treating the symptoms of active mycobacterial infection. The present method provides key advantages over conventional anti-mycobacterial therapies that often require repeated administration of the therapeutic agent over periods of several months. In a general sense, the method involves the administration of a limited number of doses of a composition comprising at least a first surface active agent (e.g., surfactants and detergents) compound in an amount and for a time sufficient to improving patient compliance in treating the symptoms of an active mycobacterial infection. In certain applications of this method, the antimicrobial surface active agent composition may be given to the patient in a single dose.

Alternatively, the patient may receive as few as 2 or three doses of an antimycobacterial therapeutic administered over a period of time. Such time periods ranging of from about 1 or two hours to about 1 or two weeks are particularly contemplated to be useful in the short-term antimycobacterial therapy as described herein. In any event, the dosage regimen and administration of the anti-mycobacterial surfactant composition will be substantially shorter in duration than the existing multi-month dosing regimens of conventional antitubercular antibiotics.

In a seventh embodiment, the invention provides a means for substantially eliminating the presence of virulent mycobacteria in the body or at least a first body tissue of an animal that has, is suspected of having, or is at risk for developing, an active mycobacterial infection within the body or body tissue of the individual. Such a method generally comprises the administration of a single dose of a pharmaceutical formulation comprising, consisting essentially or, or consisting of, at least a first anti-mycobacterial surfactant compound in an amount sufficient to substantially eliminate the presence of virulent mycobacteria from the body or a body tissue of such an animal within a period of from about a few hours to about one or two days following administration of the single dose regimen. In certain circumstances, the number of virulent microorganisms within the animal may be decreased by at least 75%, at least 80%, at least 85%, at least 90%, or even at least 95% or greater upon administration of such single-dose compositions. In other embodiments, even greater reduction in the number of infectious microorganisms may be achieved upon administration of a single dose of the therapeuticum—for example, in certain instances, the inventors contemplate that a single administration of the antimicrobial surfactant may reduce the number of virulent bacterial cells by at least 98%, at least 99%, or even at least 99.9%, or 99.99% or greater, as measured by conventional microbial enumeration methods and/or protocols.

In certain embodiments, the significant reduction in the number of viable bacterial cells upon administration of the disclosed surfactant compositions may be observed as soon as a few hours post-administration, and may be of sufficient reduction to slow or even halt the spread of the infection in the affected individual.

In the practice of any of the methods of the invention, the composition administered to the mammal may also further optionally comprise at least a second surfactant, or may also further comprise at least a first antimicrobial compound.

In some embodiments, an advantage may be achieved by formulating the composition to comprise at least two distinct surfactant compounds, each present in the composition in an amount effective to treat, prevent, or lessen the symptoms of mycobacterial infection in the individual. Similarly, in other embodiments, the inventors contemplate that a desirable outcome may be achieved by formulating the composition such that it comprise at least two surfactant compounds, and at least one antimicrobial compounds, each present in the composition in an amount effective to treat, prevent, or lessen the symptoms of mycobacterial infection in the individual.

In other embodiments, additional advantages may be achieved by formulating the therapeutic composition to comprise at least two distinct surfactant compounds, and at least two distinct antimicrobial compounds, each present in the composition in an amount effective to treat, prevent, or lessen the symptoms of mycobacterial infection in the individual.

Owing to the particularly insidious nature of mycobacterial infections, and their potential to cause disease outbreaks in large numbers of individuals, the inventors also contemplate that formulation of the composition to comprise at least two distinct surfactant compounds and at least one or more conventional “second-” or “third-line” antimicrobial compound(s) to treat or prevent a bacterial infection, or to lessen or ameliorate one or more symptoms associated with mycobacterial infection in a given individual.

In each of the aforementioned methods, the compositions may be formulated to comprise one or more surface-active compounds, including, but not limited to, surfactants having glucoside or maltoside residues, with C₈-C₁₂ fatty acid sidechain-containing compounds being particularly preferred due to their ability to interact with mycolic acid hydrophobic chains, thus more effectively reversing the aggregated (cord) state of populations of mycobacterial cells.

Exemplary surface-active compounds include, but are not limited to, non-ionic surfactants, including, for example, surfactant compounds selected from the group consisting of n-octanoylsucrose, n-nonanoylsucrose, n-decanoylsucrose, n-undecanoylsucrose, n-dodecanoylsucrose, n-heptyl-β-D-glucoside, n-heptyl-β-D-maltoside, n-heptyl-β-D-maltopyranoside, n-heptyl-β-D-glucopyranoside, n-heptyl-β-D-thioglucoside, n-heptyl-β-D-thiomaltoside, n-heptyl-β-D-thiomaltopyranoside, n-heptyl-β-D-thioglucopyranoside, n-octyl-β-D-glucoside, n-octyl-β-D-maltoside, n-octyl-β-D-maltopyranoside, n-octyl-β-D-glucopyranoside, n-octyl-β-D-thioglucoside, n-octyl-β-D-thiomaltoside, n-octyl-β-D-thiomaltopyranoside, n-octyl-β-D-thioglucopyranoside, n-nonyl-β-D-glucoside, n-nonyl-β-D-maltoside, n-nonyl-β-D-maltopyranoside, n-nonyl-β-D-glucopyranoside, n-nonyl-β-D-thioglucoside, n-nonyl-β-D-thiomaltoside, n-nonyl-β-D-thiomaltopyranoside, n-nonyl-β-D-thioglucopyranoside, n-decyl-β-D-glucoside, n-decyl-β-D-maltoside, n-decyl-β-D-maltopyranoside, n-decyl-β-D-glucopyranoside, n-decyl-β-D-thioglucoside, n-decyl-β-D-thiomaltoside, n-decyl-β-D-thiomaltopyranoside, n-decyl-β-D-thioglucopyranoside, n-undecyl-β-D-glucoside, n-undecyl-β-D-maltoside, n-undecyl-β-D-maltopyranoside, n-undecyl-β-D-glucopyranoside, n-undecyl-β-D-thioglucoside, n-undecyl-β-D-thiomaltoside, n-undecyl-β-D-thiomaltopyranoside, n-undecyl-β-D-thioglucopyranoside, n-dodecyl-β-D-glucoside, n-dodecyl-β-D-maltoside, n-dodecyl-β-D-maltopyranoside, n-dodecyl-β-D-glucopyranoside, n-dodecyl-β-D-thioglucoside, n-dodecyl-β-D-thiomaltoside, n-dodecyl-β-D-thiomaltopyranoside and n-dodecyl-β-D-thioglucopyranoside, either alone or in combination with one or more additional surfactants and/or antimicrobial compounds.

Alternatively, the inventors contemplate that the use of one or more commercial formulations of pulmonary surfactants may also be utilized in the practice of the disclosed methods and in the formulation of the disclosed medicaments. Examples of animal-derived pulmonary surfactants that are contemplated to be useful in the present invention include, but are not limited to, Alveofact® and Infasurf® (both extracts from bovine lung lavage fluids); Curosurf® (extracted from macerated porcine lung tissue); Survanta® (extracted from macerated bovine lung and supplemented with additional DPPC, palmitic acid and tripalmitine), and other surfactant compositions suitably formulated for administration to a mammal.

Examples of synthetic pulmonary surfactant compounds that may be utilized in the formulating antimycobacterial compositions include, but are not limited to, Exosurf® (a mixture of DPPC with hexadeconal and tyloxapol added as spreading agents); Pumactant® (a mixture of DPPC and PG; also known as “Artificial Lung Expanding Compound” or ALEC); KL-4 (a mixture of DPPC, palmitoyloleoyl phosphatidylglycerol, and palmitic acid, combined with a 21 amino acid synthetic peptide that mimics the structural characteristics of SP-B); and Venticute® (a mixture of DPPC, PG, palmitic acid and recombinant SP-C).

In exemplary embodiments, the surfactant compound may be selected from non-ionic surfactants, including for example, those surfactants selected from the group consisting of n-dodecyl-β-D-maltoside (“DDM”), n-octyl-β-D-maltoside (“OM”) and n-octyl-β-D-glucoside (“OG”).

One or more conventional antimicrobial compound(s) may also be added to the anti-mycobacterial formulations described herein. Exemplary conventional compounds include for example (but are not limited to), antibiotics, antivirals, antihelminths, antibacterials, antifungals, antimycotics, antimycobacterials, and such like. Exemplary antibiotics known to be effective in the treatment of M. tuberculosis infections include, but are not limited to, capreomycin, cycloserine, ethambutol, ethionamide, isoniazid, kanamycin, levofloxacin, moxifloxacin, ofloxacin, pyrazinamide, rifabutin, rifampin, rifapentine, streptomycin, tobramycin, p-aminosalicylic acid, clofazimine, imipenem, linezolid and amoxicillin.

Preferably the compositions of the present invention are suitably formulated for administration to mammals, and to human beings in particular. The compositions may also be formulated for use in veterinary medicine, and for administration to affected mammalian species including primates, domesticated livestock, small mammals, pets, and such like.

In the formulation of such therapeutic and prophylactic reagents, the anti-bacterial compositions of the present invention may comprise, consist essentially of, or consist of a first surfactant compound in an amount from about 0.01 mg/ml to about 10.0 mg/ml; in multi-drug formulations, the anti-mycobacterial compositions may also comprise at least a second distinct surfactant compound that is also present in the formulation in an amount from about 0.01 mg/ml to about 10.0 mg/ml.

In exemplary embodiments, it may be desirable that the selected surfactant compounds are each present in the pharmaceutical formulation in an amount of from about 0.10 mg/ml to about 1.0 mg/ml, or alternatively, present in the formulation in an amount of from about 0.3 mg/ml to about 0.8 mg/ml.

In the formulation of combination therapeutic reagents, the anti-mycobacterial compositions of the present invention may further comprise a first antibiotic in an amount from about 0.01 mg/ml to about 10.0 mg/ml; in multi-drug formulations, the anti-mycobacterial compositions may also comprise at least a second distinct antibiotic compound that is also present in the formulation in an amount from about 0.01 mg/ml to about 10.0 mg/ml.

In exemplary embodiments, it may be desirable that the selected surfactant compounds (and any optional antibiotics) are each present in the pharmaceutical formulation in an amount of from about 0.10 mg/ml to about 1.0 mg/ml, or alternatively, present in the formulation each in an amount of from about 0.3 mg/ml to about 0.8 mg/ml.

The pharmaceutical compositions of the present invention will preferably be formulated for administration to the recipient by conventional administration routes, with direct introduction to the lungs via inhalation, vaporization, aerosol injection, or nebulization being particularly preferred. Formulations of pharmaceuticals for administration to the lungs have been broadly described in the literature, and known to those of skill in the art. Examples of various means for delivering one or more compositions to the lung surface include, U.S. Pat. Nos. 4,852,561, 4,955,371, 5,277,913, 5,505,194, 5,642,728, 5,692,493, 5,934,273, 6,250,300, 6,360,743, 6,406,745, 6,567,686, 6,645,466, 6,716,414, 6,849,269, 6,967,017, 6,971,385, 6,984,404, 7,018,618, 7,063,748, 7,186,401, and 7,223,381, each of which is specifically incorporated herein by reference in their entirety.

Alternatively, the composition may be directly administered to one or more tissues or tissue sites within the body of an individual. Such modes of administration are well-known to those of skill in the art, and include, for example, directed administration to at least a first lung tissue or at least a first site within the thoracic cavity of the individual selected to receive treatment.

In the practice of the therapeutic and prophylactic treatment regimens of the invention, the inventors contemplate that the anti-tubercular compositions will preferably be administered to the selected mammalian recipient in a single dose, or in a plurality of successive doses, for a period of time of from about a one time administration, up to a dosing regimen of from between about two and about six times per day, with the length of treatment preferably being on the order of from about one day to about one week or even to and including a period of administration lasting from about two weeks to about six months or more. In particular embodiments, the administration may be restricted to a single one-time dose, or to administration of two or three doses given over a relatively short period of time (e.g., from a few hours to a few days).

In another embodiment, the invention provides methods for preventing the onset of TB symptoms in a mammal that has been exposed to one or more virulent strains of Mycobacterium tuberculosis bacteria, and that shows a determinable count of the bacterium in its system, but has not yet demonstrated the classical clinical symptoms of active tubercular infection. Such a method generally involves at least the steps of: (a) determining the presence of Mycobacterium tuberculosis in the sputum of an individual exposed to the Mycobacterium tuberculosis strain; (b) administering to the individual so exposed a therapeutically effective amount of at least one of the anti-tubercular compositions described herein in an amount, and for a time sufficient, to prevent the onset of active tubercular symptoms in the individual; and (c) determining the substantial absence of virulent Mycobacterium tuberculosis bacterial cells in the sputum of the treated individual at a time subsequent to initial administration of the anti-tubercular composition.

In another embodiment, the invention also provides a method for inducing prophylaxis against mycobacterial infection in a mammal that subsequently may be exposed to a pathogenic strain of Mycobacterium tuberculosis. Such a method generally involves at least the step of administering to a mammal in need thereof, a prophylactically-effective amount of one or more of the anti-tubercular compositions disclosed herein, in an amount, and for a time sufficient to prevent, reduce, delay, or ameliorate the symptoms of the onset of TB symptoms in the mammal upon its subsequent exposure to one or more virulent strains of Mycobacteria, including for example, Mycobacterium tuberculosis.

In one embodiment, the invention provides a method of treating or ameliorating the symptoms of a mycobacterial infection in a mammal, the method comprising at least the step of: administering to the mammal a therapeutically-effective amount of a composition that comprises, consists essentially of, or consists of, at least a first surfactant for a time sufficient to treat or ameliorate the symptoms of the infection in the mammal.

The infection may be caused by any mycobacterial species, with Mycobacterium africanum, Mycobacterium avium, Mycobacterium avium paratuberculosis, Mycobacterium avium silvaticum, Mycobacterium avium hominissuis, Mycobacterium avium colombiense, Mycobacterium botniense, Mycobacterium bovis, Mycobacterium canetti, Mycobacterium caprae, Mycobacterium haemophilum, Mycobacterium heckeshornense, Mycobacterium intracellulare, Mycobacterium kansasii, Mycobacterium leprae, Mycobacterium microti, Mycobacterium paratuberculosis, Mycobacterium pinnipdeii, Mycobacterium tuberculosis, Mycobacterium ulcerans and Mycobacterium xenopi being particularly contemplated as amenable to treatment using one or more of the methods and pharmaceutical anti-mycobacterial formulations as described herein.

The anti-mycobacterial compositions of the present invention will preferably comprise, consist essentially of, or consist of, a first surfactant compound in an amount from about 0.001 mg/ml to about 1000.0 mg/ml, or more preferably in an amount of from about 0.01 mg/ml to about 100.0 mg/ml, and more preferably still, in an amount of from about 0.1 mg/ml to about 10.0 mg/ml.

The compositions of the present invention may be formulated for administration to a selected subject by any conventional administration means, but it is preferable that the composition be provided to the selected subject either by inhalation, vaporization, aerosol injection, nebulization, or direct administration to one or more tissue sites and/or body cavities of the affected individual. In certain embodiments, it may be desirable to apply the composition directly to the site of active infection, including, for example, to one or more sites within the thoracic cavity of the individual, or to one or more sites directly in the lung of the individual.

The compositions are preferably administered to the selected individual in any amount and for any duration as required to achieve the desired therapeutic and/or prophylactic result. However, the inventors contemplate that administration of the pharmaceutical formulations disclosed herein will likely be on the order of from one to about six times per day, alternatively, on the order of from one to about four times per day, or even on the order of from one to about twice a day. Similarly, the contemplated length of the drug regimen will be significantly shorter than the conventional eight- to twelve-month protocols, and may be as short a period as from one day to about three months. Preferably, the length of time required for administration of the composition will be as short a period as from one day to about one month, or even as short as a period of from one day to about fourteen days, or even as short as a period of from one day to about seven days.

Turning to another aspect, the invention also provides methods for preventing the onset of TB symptoms in an individual exposed to one or more infectious strains of Mycobacterium tuberculosis bacteria, and showing a determinable count of said bacterium, but not yet experiencing the clinical symptoms of TB. In an overall sense, this method generally involves at least the steps of: (a) determining the presence of M. tuberculosis in the sputum of an individual exposed to M. tuberculosis; (b) administering to the individual a therapeutically-effective amount of a composition that comprises at least a first surfactant compound for a time sufficient to prevent the onset of clinical symptoms of TB in the individual; and (c) determining the substantial absence of viable M. tuberculosis cells in the sputum of the individual following administration of the composition, wherein the substantial absence of viable M. tuberculosis cells in the sputum following administration is effective to prevent the exposed individual from developing one or more clinical symptoms of TB.

The invention further provides a method for inducing prophylaxis against TB or other mycobacterial infections in a mammal that subsequently may be exposed to at least a first pathogenic species of a member of the genus, Mycobacterium. This method generally comprises or consists essentially of: at least the step of administering to a mammal a first prophylactically-effective amount of a pharmaceutical composition that comprises at least a first pulmonary surfactant for a time sufficient to prevent the onset of TB symptoms in the mammal upon subsequent exposure to a pathogenic strain of Mycobacterium tuberculosis. This method may also further optionally comprise the additional step of subsequently administering to the mammal at least a second prophylactically-effective amount of a pharmaceutical composition that comprises an anti-mycobacterial compound. The method may also further optionally comprise the additional step of subsequently administering to the mammal another dose of at least one anti-mycobacterial composition.

In such methods, the second prophylactically-effective amount (“dose”) of a composition that comprises one or more of the surface-active ingredients described herein, may be provided to the mammal one or more weeks following administration of the initial dose, and may be followed by one or more successive administrations as required to achieve the desired clinical effects. The determination of individual dose size, duration of therapy, the need for concomitant co-therapy using a second biologically-active compound, as well as all other dosing considerations is considered to be well within the perview of the skilled medical practitioner involved in the practice of the invention, and is not further detailed herein.

The present invention also provides pharmaceutical compositions that comprise at least one anti-mycobacterial surfactant formulated for administration to an animal (either systemically, or by direct administration to one or more body cavities, organs, tissues, and/or cells for use in the therapy of mycobacterial infections in general, and in the therapy of M. tuberculosis infection, and other species pathogenic to man in particular. Exemplary administration sites include, for example, but are not limited to, systemic administration to the circulatory system, injection into one or more cells or tissue sites, or to one or more organs or tissue types such as a lung or a lung tissue of a mammal, or to the thoracic cavity of the animal. Alternatively, the compositions may be formulated as described infra into formulations that may be administered orally, intranasally, or by inhalation or infiltration into the pulmonary system of the animal.

The invention also provides for the use of such surfactant-based compounds in the manufacture of one or more medicaments for treating, preventing, or ameliorating one or more clinical symptoms of mycobacterial infections in general, and in the treatment, prevention, or amelioration of clinical symptoms in M. tuberculosis infections in particular.

Use of a composition comprising at least a first surfactant formulated for administration to the lung or lung tissue of a mammal in the manufacture of a medicament for treating or ameliorating the symptoms of a mycobacterial infection (and particularly for a M. tuberculosis infection) is also provided by the invention, as is the use of such compositions in the treatment of human beings, companion animals, domesticated livestock, exotics, and other mammals under the care of a physician, veterinarian, or other medical practitioner or healthcare provider.

In contemplation of these uses of the disclosed compositions, the invention also provides for the manufacture of medicaments that further comprise one or more antibacterial or antimicrobial agents effective against one or more bacterial species (including, for example, mycobacteria such as M. tuberculosis), or a second surfactant/detergent/surface-active composition that exerts bactericidal and/or bacteriostatic action upon at least a first susceptible strain of a first pathogenic bacterials species, and that of a pathogenic mycobacterial species such as M. tuberculosis, in particular. While the practice of the invention is contemplated to have the most relevant and most immediate utility in the treatment of M. tuberculosis infections, the inventors in no way intend for the scope of the present disclosure to be interpreted as being limited to the treatment of only M. tuberculosis, nor do they intend for the disclosure to be interpreted as being limited only to the administration of a specific surface active agent as enumerated herein. Indeed, the inventors believe that the present invention may find broad applicability in a variety of antimicrobial treatment/prophylaxis regimens, including other bacterial species, as well as other non-bacterial pathogens.

### BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, and FIG. 1F show the effect of OG on MTB preparation. The Erdman strain of MTB was grown in a clear Eppendorf tube until extensive aggregation of the bacteria had occurred as shown in FIG. 1A and FIG. 1B. Samples (0.5 ml) were added to clear Eppendorf tubes containing 5 mg of OG (1% wt/vol.), and vortexed for ˜15 sec. Samples were added to glass slides after incubation for 30 min (FIG. 1C and FIG. 1D) and 24 hr (FIG. 1E and FIG. 1F) with surfactant. The 2× views represent nearly the entire sample on the slide. The specimens were air dried and stained using the Kinyon procedure. The OG rapidly reversed aggregation of MTB, and the 50× image shows that over 90% of the bacteria are eliminated within 24 hours of treatment.

FIG. 2A and FIG. 2B illustrate the effects of DDM on MTB. The bacteria were grown as described in FIG. 1A-FIG. 1F and 0.5 ml added to Eppendorf tubes containing 5 mg of DDM (1% wt/vol.). After two hour's exposure to the surfactant, the preparation was sampled for imaging. The rapid loss of cording (FIG. 2A) and the elimination of most of the bacteria (FIG. 2B) indicate that surfactants that are relatively soluble in water (i.e., high CMC values) are very effective in disrupting the aggregation of MTB, act as a bacteriocide, and may offer a new and effective treatment option for MTB.

FIG. 3 shows the histological assessment of the toxicity of DDM and OG (1% each) introduced as an aerosol into mice lungs as compared to controls and described in Section 5.3 infra. There are no identifiable histopathological differences between the control and experimental mice lung, liver and spleen tissues. These results show that these surfactants are well tolerated by the lungs.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of the present disclosure.

### Effects of Surfactants on MTB Cord Formation

Electron microscopy studies of MTB show that TDM is extraordinarily hard and is approximately five-fold thicker than any other known lipid bilayer. This unique outer shield forms an effective impenetrable barrier to the host's immune system, but also acts as MTB's “Achilles' heel.” For largely unknown reasons, MTB may form cords in a sub-population of patients that are related to the active form of the disease. The results presented herein demonstrate that certain surfactants effectively reverse the corded state of MTB and probably remove most of the mycolic acid protective coat from the bacteria. Consequently, the pathogenic state of the bacteria is reduced and the bacteria are susceptible to clearance by the immune system.

The lung surfactants may have a similar action as detergents which have been shown to prevent MTB cell adhesion as shown below and to reverse the aggregation process which is driven by the hydrophobic interactions between cells.

### Antitubercular Compounds and Pharmaceutical Compositions

The unique characteristics of tubercular infections suggest that application of a pharmaceutical formulation comprising bioeffective amounts of one or more surfactant compound(s), either alone, or in combination with one or more other anti-bacterial agents offers effective, inexpensive and short-term alternatives for treatment of the disease and/or amelioration of one or more of its clinical symptoms. These compositions may also provide for the first time an effective means for prophylaxis of the disease in populations susceptible to their exposure and resulting infection.

In a first embodiment, the invention provides compositions comprising, consisting of, or consisting essentially of, one or more of the anti-MTB surfactant compounds disclosed herein.

### Surface Active Compounds and Compositions Comprising Them

Pulmonary surfactant is a complex and highly surface-active material composed of lipids and proteins, and which is found in the fluid lining the alveolar surface of mammalian lungs.

The alveolar surface of the human lung is covered with primarily two surfactants—dipalmitoylphosphatidylcholine (DPPC; 1,2-dipalmitoyl-sn-3-glycerophosphoryl choline) (which accounts for approximately 50 to 70% by weight of the total pulmonary surfactant in normal human adults) and phosphatidylglycerol (PG)—which have the important function of reducing surface tension so that breathing is possible. It is noteworthy that these two surfactants are routinely administered to premature babies by direct application into their trachea to reduce respiratory distress syndrome (RDS).

Exemplary surfactant compounds include, but are not limited to, n-octanoylsucrose, n-nonanoylsucrose, n-decanoylsucrose, n-undecanoylsucrose, and n-dodecanoylsucrose; n-heptyl-β-D-glucosides including for example, but not limited to, n-heptyl-β-D-glucoside, n-heptyl-β-D-maltoside, n-heptyl-β-D-maltopyranoside, and n-heptyl-β-D-glucopyranoside; n-heptyl-β-D-thioglucosides, including for example, but not limited to, n-heptyl-β-D-thioglucoside, n-heptyl-β-D-thiomaltoside, n-heptyl-β-D-thiomaltopyranoside and n-heptyl-β-D-thioglucopyranoside; n-octyl-β-D-glucosides including for example, but not limited to, n-octyl-β-D-glucoside, n-octyl-β-D-maltoside, n-octyl-β-D-maltopyranoside, and n-octyl-β-D-glucopyranoside; n-octyl-β-D-thioglucosides, including for example, but not limited to, n-octyl-β-D-thioglucoside, n-octyl-β-D-thiomaltoside, n-octyl-β-D-thiomaltopyranoside and n-octyl-β-D-thioglucopyranoside; n-nonyl-β-D-glucosides including for example (but not limited to), n-nonyl-β-D-glucoside, n-nonyl-β-D-maltoside, n-nonyl-β-D-maltopyranoside, and n-nonyl-β-D-glucopyranoside; n-nonyl-β-D-thioglucosides, including for example (but not limited to), n-nonyl-β-D-thioglucoside, n-nonyl-β-D-thiomaltoside, n-nonyl-β-D-thiomaltopyranoside and n-nonyl-β-D-thioglucopyranoside; n-decyl-β-D-glucosides including for example (but not limited to), n-decyl-β-D-glucoside, n-decyl-β-D-maltoside, n-decyl-β-D-maltopyranoside, and n-decyl-β-D-glucopyranoside; n-decyl-β-D-thioglucosides, including for example (but not limited to), n-decyl-β-D-thioglucoside, n-decyl-β-D-thiomaltoside, n-decyl-β-D-thiomaltopyranoside and n-decyl-β-D-thioglucopyranoside; n-undecyl-β-D-glucosides including for example (but not limited to), n-undecyl-β-D-glucoside, n-undecyl-β-D-maltoside, n-undecyl-β-D-maltopyranoside, and n-undecyl-β-D-glucopyranoside; n-undecyl-β-D-thioglucosides, including for example (but not limited to), n-undecyl-β-D-thioglucoside, n-undecyl-β-D-thiomaltoside, n-undecyl-β-D-thiomaltopyranoside and n-undecyl-β-D-thioglucopyranoside; n-dodecyl-β-D-glucosides including for example (but not limited to), n-dodecyl-β-D-glucoside, n-dodecyl-β-D-maltoside, n-dodecyl-β-D-maltopyranoside, and n-dodecyl-β-D-glucopyranoside; n-dodecyl-β-D-thioglucosides, including for example (but not limited to), n-dodecyl-β-D-thioglucoside, n-dodecyl-β-D-thiomaltoside, n-dodecyl-β-D-thiomaltopyranoside and n-dodecyl-β-D-thioglucopyranoside; as well as synthetic surfactant compounds such as Exosurf® (a mixture of DPPC with hexadeconal and tyloxapol added as spreading agents); Pumactant® (a mixture of DPPC and PG; also known as “Artificial Lung Expanding Compound” or ALEC); KL-4 (a mixture of DPPC, palmitoyloleoyl phosphatidylglycerol, and palmitic acid, combined with a 21 amino acid synthetic peptide that mimics the structural characteristics of SP-B); and Venticute® (a mixture of DPPC, PG, palmitic acid and recombinant SP-C).

Examples of animal-derived surfactants include, but are not limited to, Alveofact® and Infasurf® (both extracts from bovine lung lavage fluids); Curosurf) (extracted from macerated porcine lung tissue); and Survanta® (extracted from macerated bovine lung and supplemented with additional DPPC, palmitic acid and tripalmitine).

The pharmaceutical compositions disclosed herein may be delivered via oral administration to an animal, and as such, these compositions may be formulated with an inert diluent or with an assimilable edible carrier, or they may be enclosed in hard- or soft-shell gelatin capsule, or they may be compressed into tablets, or they may be incorporated directly with the food of the diet

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Alternatively, the pharmaceutical compositions disclosed herein may be administered parenterally, intravenously, intramuscularly, or even intraperitoneally as described in U.S. Pat. No. 5,543,158, U.S. Pat. No. 5,641,515 and U.S. Pat. No. 5,399,363 (each specifically incorporated herein by reference in its entirety). Solutions of the active compounds as free-base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermic lysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Examples of pharmaceutically-acceptable salts may include the acid-addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The administration of the pharmaceutical compositions by intranasal sprays, inhalation, and/or other aerosol delivery vehicles is also contemplated. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety), and delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In addition to the methods of delivery described above, the following techniques are also contemplated as alternative methods of delivering anti-mycobacterial surfactant-based compositions. Sonophoresis (i.e., ultrasound) has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), transdermal matrices (U.S. Pat. No. 5,770,219 and U. S. Pat. No. 5,783,208) and feedback-controlled delivery (U.S. Pat. No. 5,697,899), each specifically incorporated herein by reference in its entirety.

### Administration of Antitubercular Compounds

In particular embodiments the disclosed anti-MTB active agents will be formulated for systemic or local administration to a mammal, and in particular, a human, that has, is suspected of having, or is at risk for developing a mycobacterial infection, and in particular one or more diseases that are indicative of an infection having Mycobacterium tuberculosis as a causative agent.

The active ingredients, compounds, and compositions of the invention are preferably formulated for administration to one or more cells, tissues, or organs in a mammal receiving such treatment, or for systemic administration of one or more anti-MTB compounds to a human, or to an animal under veterinary care. The formulations may also be prepared in one or more containers or vials for commercial sale, or may be provided in combination with one or more delivery or administration means, such as for example, an aerosol (including monodisperse and polydisperse modalities), an inhalant, nebulizer, dry-powder inhalants, and/or other suitable devices to administer the compounds or compositions directly to the surface of a mammalian lung.

Various methodologies for delivery of pharmaceutical compositions to the lung have been developed and extensively improved in the last decade (see e.g., O'Callaghan et al., 2002).

### Commercial Formulations and Therapeutic Kits

The present invention also encompasses one or more of the disclosed anti-tubercular compositions formulated together with one or more pharmaceutically-acceptable excipients, carriers, diluents, adjuvants, buffers, and such like, packaged for commercial sale in suitable container means, into which the disclosed compositions may be packaged, either as “stock” concentrates (which can be diluted prior to administration using a sterile injectate or sterile physiological solution, or as final “working” solutions that is already diluted in a “ready-to-inject” solution. The composition may include one or more surfactant compounds, either alone, or in combination with one or more additional anti-microbial active ingredients. Therapeutic kits may also be prepared that comprise at least one of the antibacterial compositions disclosed herein and instructions for using the composition as a therapeutic agent in the treatment and/or prevention of mycobacterial infections.

The container means for such kits may typically comprise at least one vial, test tube, flask, bottle, syringe or other container means, into which the active ingredient(s) may be placed, and preferably suitably aliquotted. Where a second antimicrobial agent is also provided, the kit may also contain a second distinct container means into which this second composition may be placed. Alternatively, a plurality of antimicrobial compounds may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

Such kits may also include one or more instruction sets or protocols detailing particular method(s) of use of the anti-tubercular compositions, and may optionally further include one or more containers for additional pharmaceuticals, or other drugs that may be co-administered with the anti-MTB compounds disclosed herein.

Alternatively, one or more optional devices for delivering the pharmaceutical compositions to a patient, or to a particular site within the body of a patient may also be included. Such kits may include ready-dose sterile needle/syringes, inhalers, nebulizers, metered-dose nasal spray dispensers, etc. that may optionally be individually prepackaged and optionally sterilized for convenient commercial packaging, sale, use, transport, and administration.

Such compositions may also comprise one or more pharmaceutically-acceptable excipients, carriers, diluents, adjuvants, buffers, and/or other components useful in the preparation of medicaments suitable for systemic or local administration to a mammal having, suspected of having, or at risk for developing N1113 or a related mycobacterial infection.

Preferred animals for administration of the pharmaceutical compositions disclosed herein include mammals, and particularly humans, but other mammalian species are also contemplated to be benefited by the disclosed anti-mycobacterial compositions, including, for example, but not limited to, murines, bovines, ovines, caprines, equines, porcines, lupines, canines, felines, and non-human primates under veterinary care.

### Liposome-, Nanocapsule-, and Microparticle-Formulations

In certain embodiments, the inventors contemplate the use of biodegradable liposomes, nanocapsules, microparticles, microspheres, lipid particles, vesicles, and the like, for introduction of an antitubercular agent, or a composition comprising one or more antitubercular agents into suitable mammalian cells, tissues, organs, or infection site. In some embodiments, the antitubercular agents of the present invention may also be formulated for delivery either encapsulated in a lipid particle, a liposome, a vesicle, a nanosphere, or a nanoparticle or the like.

Such formulations may be preferred for the introduction of pharmaceutically acceptable formulations and compositions disclosed herein. The formation and use of liposomes is generally known to those of skill in the art (see for example, U.S. Pat. No. 5,741,516, U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

### Exemplary Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of clarity, the following specific terms are defined below:

As used herein, the terms “patient” and “recipient” are intended to include animals, and in particular, mammalian species such as human beings, livestock, or animals under veterinary care and/or supervision.

The term “buffer,” as used herein, refers to aqueous solutions or compositions that resist changes in pH when acids or bases are added to the solution. Solutions that exhibit buffering activity are often referred to in the art as “buffers” or “buffer solutions.” Buffers typically are able to maintain the pH of the solution within defined ranges, often for example between pH 5.5 and pH 7.5. Buffer solutions that are typically able to maintain a pH of approximately 7, are often referred to a “physiological buffers.” Exemplary biological buffers include, but are not limited to, Lactated Ringer's solution, physiological saline solution, N-(2-Acetamido)-2-aminoethanesulfonic acid (ACES); N-2-acetamido-2-iminodiacetic acid (ADA); N,N-bis(2-hydroxyethyl)-2-aminoethanesulfonic acid (BES); N,N-bis(2-hydroxyethyl)glycine (BICINE); 2-bis(2-hydroxyethyl)amino-2-(hydroxymethyl)-1,3-propanediol (BIS-TRIS); 3-(cyclohexylamino)-1-propanesulfonic acid (CAPS); 3-(cyclohexylamino)-2-hydroxy-1-propanesulfonic acid (CAPSO); 2-(cyclohexylamino)ethanesulfonic acid (CHES); (N,N-bis[2-hydroxyethyl]amino)-2-hydroxypropanesulfonic acid (DIPSO); 4-(2-Hydroxyethyl)-1-piperazinepropanesulfonic acid (EPPS); 4-(2-hydroxyethyl)piperazine-1-ethanesulfonic acid (HEPES); 4-(2-hydroxyethyl)piperazine-1-(2-hydroxypropanesulfonic acid) (HEPPSO); 2-(N-morphilino)ethanesulfonic acid (MES); 3-(N-morpholino)propanesulfonic acid (MOPS); 3-morpholino-2-hydroxypropanesulfonic acid (MOPSO); piperazine-1,4-bis(2-ethanesulfonic acid) (PIPES); piperazine-N,N′-bis(2-hydroxypropanesulfonic acid) (POPSO); [(2-hydroxy-1,1-bis(hydroxymethypethyl)amino]-1-propanesulfonic acid (TAPS); 3-(N-tris[hydroxymethyl]methylamino)-2-hydroxypropanesulfonic acid (TAPSO); 2-[(2-Hydroxy-1,1-bis(hydroxylmethyl)ethyl)amino]ethanesulfonic acid (TES); N-[tris(hydroxymethyl)methyl]glycine (TRICINE); and tris(hydroxymethyl)aminomethane) (TRIS); mixtures or derivatives thereof, as well as other biological buffers including those developed by Good et al. (1966).

Likewise, all integers and sub-ranges within a given range of measurement (e.g., concentration) are also specifically considered to fall within the scope of the present teaching. For example, where a particular range of concentration is given, for example, “between about 0.001% and about 50%” or “from about 0.001% to about 50%” or “within the range of from 0.001% to 50%,” etc., it is specifically intended that all intermediate sub-ranges (e.g., “from 0.01% to 40%”, or “from 0.02% to 20%” etc.) are explicitly included within the scope of the present invention. Likewise, all intermediate integers within a stated concentration range or sub-range are also explicitly encompassed by the present teaching. Therefore, it is understood that recitation of a concentration that falls within the range of “between about 0.001% and about 50%” (inter alia, e.g., 0.01%, 0.1%, 1.0%, 2%, 10%, 23%, 31.5%, 42.15%, 48.99%, etc.) implicitly fall within the scope of the present teaching and the subject matter claimed herein. Likewise, the present specification encompasses both open-ended (e.g., “at least 1%,” “at least 1.5%,” “less than about 2%,” “not more than 5 percent” etc.), as well as all closed-ended sub-ranges within a stated numerical range (e.g., the sub-ranges “between about 0.01% and about 20%” or “between about 0.01% and about 33%,” or “from approximately 0.01% to approximately 40%,” each implicitly falls within the numerical range “from about 0.01% to about 50%.”

In the context of the invention, the term “about” is given its ordinary meaning of “approximately.” Thus, the term “about 1 week” is intended to mean a period of time of approximately 7 days (equivalent to “approximately 168 hours”), which may, of course, be slightly longer than, or slightly shorter than the exact stated numerical amount. Likewise, the phrase “at least about X days” may be used to describe an interval of time that is “approximately,” “nearly,” or “almost” ‘X’ days in length or duration, but which need not necessarily be “X” days exactly. Such a time interval may be slightly less or slightly more than the absolute numerical value of “X” itself. Such understanding of the terms about and approximately are within the general knowledge of the skilled artisan, and the foregoing example is provided only to illustrate the “flexibility” the adverbs “about” and/or “approximately” render to the nouns that they modify.

In the context of the invention, a period of “about 20-30 days” is understood to be inclusive of periods that are about 20 days, about 21 days, about 22 days, about 23 days, about 24 days, about 25 days, about 26 days, about 27 days, about 28 days, about 29 days, or about 30 days, and even is inclusive of periods that may be on the order of about 19 days, or even about 31 days, to and including the fractional intervals of time within the stated range(s).

In the context of the invention, a period of “about 30-40 days” is understood to be inclusive of periods that are about 30 days, about 31 days, about 32 days, about 33 days, about 34 days, about 35 days, about 36 days, about 37 days, about 38 days, about 39 days, or about 40 days. The term “about 30-40 days” is also implicitly inclusive of periods of time that may be about 28 or 29 days, or even about 41 or 42 days, to and including the fractional intervals of time within the stated range(s).

As used herein, the term “comprising” and its cognates are used in their inclusive sense; that is, equivalent to the term “including” and its corresponding cognates. The articles “a,” “an,” and “the” explicitly include plural references unless the context clearly dictates otherwise.

### EXAMPLES

The following examples are included to demonstrate preferred embodiments of the invention. It should be appreciated by those of skill in the art that the techniques disclosed in the example which follows represents techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effects of Surfactants on Corded State of MTB

In vitro studies described in this example have established that TB bacteria exposed to the disclosed surfactant compositions (at 1% wt/vol. concentration of the active ingredient) for 2 hr showed significant destruction and disappearance of the bacteria documented by phase-contrast microscopy, conventional light microscopy with acid-fast staining and electron microscopy (EM). After 24 hr incubation, the decrease in the corded state was even more pronounced: the high-power view phase contrast microscopy clearly demonstrated strong bactericidal effect of such exposure.

### Experimental Protocol

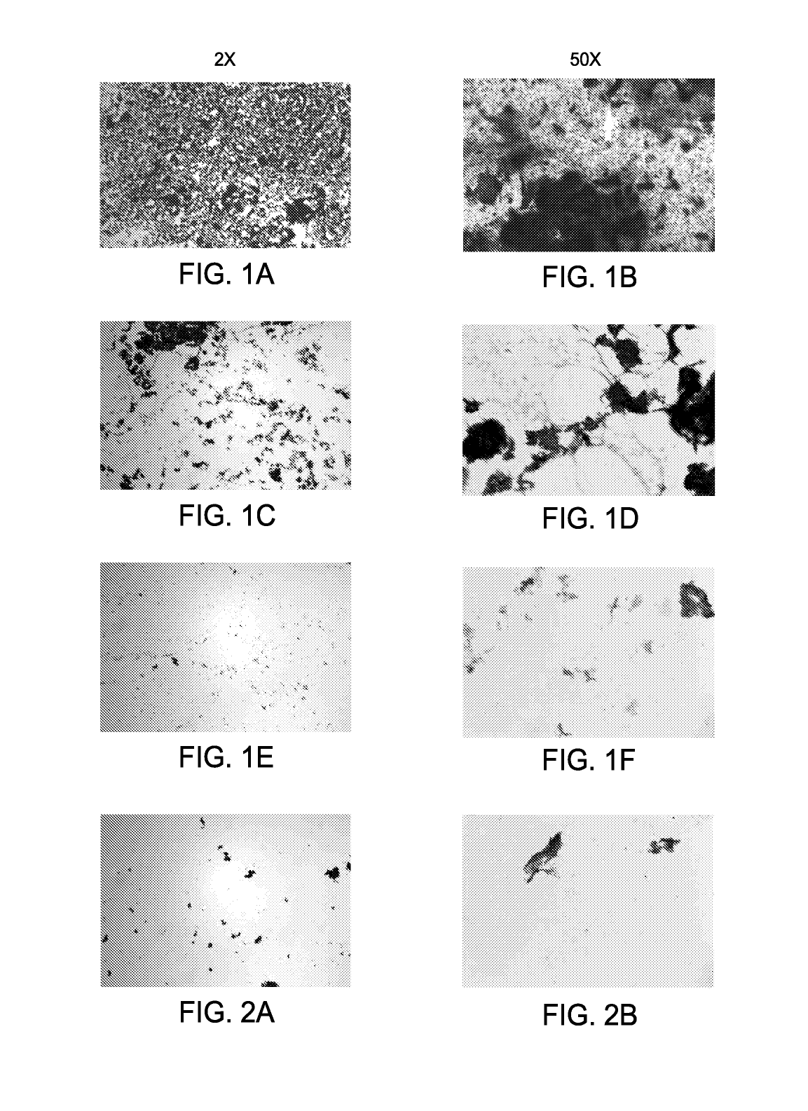

The inventors have studied the effect of surfactants on the corded state of MTB (FIG. 1). These studies establish proof that the corded state of the bacteria can be reversed. The incubation of the Erdman strain of MTB in PBS growth medium for two weeks results in extensive cording (FIG. 1A). A 50× microscopic field of the cultured bacteria shows the serpentine characteristics of the cords, as well as numerous bacteria in the background. The 2× image of the preparation shows a major reduction in the number of the cords upon addition of 1% (wt/vol.) OG to the bacterial preparation for 30 min immediately prior to sampling (cf. FIG. 1A and FIG. 1C) and a comparison of the higher power views (FIG. 1B and FIG. 1D) also shows significant loss in the number of bacteria in the background. The decrease in the corded state after 24 hr of incubation is more pronounced (cf. FIG. 1A and FIG. 1E), with the higher power views clearly demonstrating that the surfactant also acts as a bacteriocide (cf. FIG. 1B and FIG. 1F).

The in vitro effect of OG on MTB raises the issue of the mechanism of clinical stage of the disease in which cavities form usually in the apical regions of the lungs in the presence of surfactants (e.g., DPPC and PE). In this regard, it has been shown that 1% DPPC added to a preparation of corded MTB did not reverse cording over the time period of the study. The greater proficiency of OG over DPPC for the rapid reversal of the aggregated state is likely related to its relatively larger critical micelle concentration (CMC)—10⁻² M vs. that of DPPC (10⁻¹⁰ M). The much higher solution concentration of OG permits rapid interaction of the surfactant molecules with the ˜C₉₀ fatty acid chains buried in the matrix of the cord which effectively resist penetration of the large DPPC liposomes.

It appears that these surfactants (DPPC and PG) which coat the epithelium of the lung are sufficient to initially inhibit cording thereby affording a sufficient time frame for the immune system to effectively control the disease in most individuals. The action of these surfactants therefore may be related to the latent form of the disease that affects most people exposed to MTB.

The belief that surfactants that have higher solution concentration (related to their higher CMC) are more active in reversing cording was also examined in this study. For example, n-dodecyl-β-D-maltoside (C₁₂) fatty acid in an ester linkage to two cyclic sugar residues instead of the one associated with OG was investigated. The C₁₂ fatty acid affords a more hydrophobic tail than does the C₈ in OG and therefore is expected to interact more proficiently with mycolic acid's hydrophobic chain and thereby more effectively reverse the aggregated state of the bacteria.

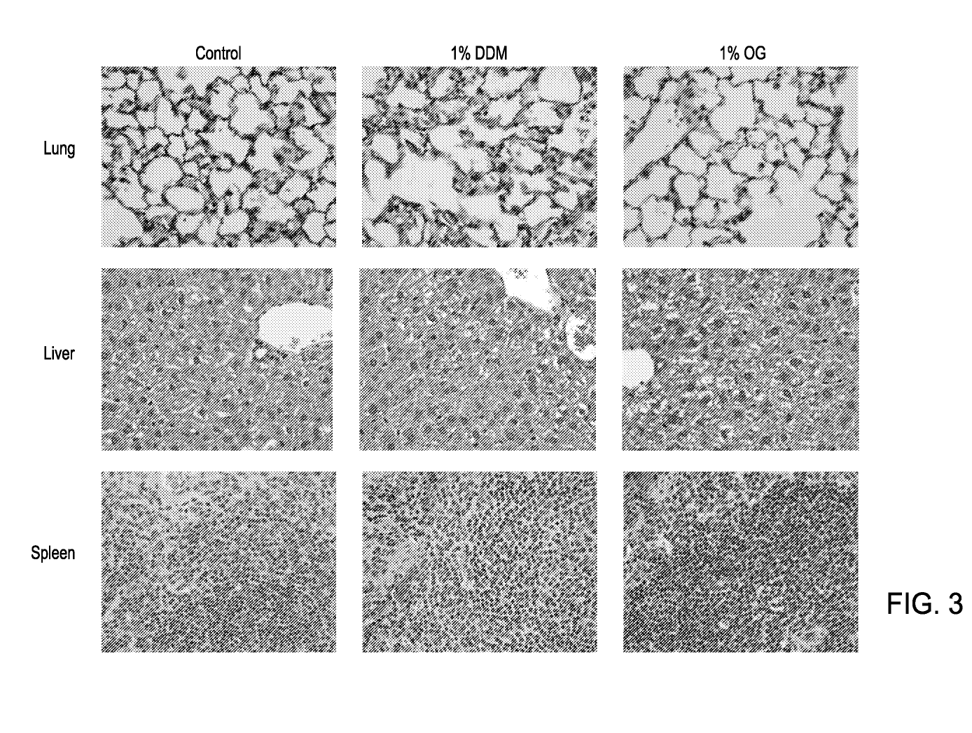

The MTB was exposed to DDM (1% wt./vol.) for 2 hr before sampling for microscopic imaging (FIG. 2A and FIG. 2B). The surfactant appears to reduce cording to a similar extent of OG after 24 hr exposure (cf. FIG. 2E and FIG. 2F to FIG. 2A and FIG. 2B). It is worth noting that both surfactants were bactericidal (cf. FIG. 1B with FIG. 1F and FIG. 2B).

This study establishes that MTB cords can be reversed by a class of surfactants with relatively high CMC values ≧˜10⁻² M. The process is rapid and nearly complete within 24 hr of exposure. In contrast, the natural lung surfactants DPPC and PG have very low CMC values and, consequently, may not have sufficient solution concentration to reverse formation of the huge macrocellular aggregates of MTB cells.

It has been shown that MTB will grow in the presence of 0.3% OG but are not surrounded by their extensive coat of mycolic acid. This result suggested that surfactants may also be effective in the treatment of MTB at even lower concentrations.

The proficiency of these surfactants with high CMC valves (e.g., OG and DDM) in cord reversal and their bactericidal behavior may offer a new and proficient therapeutic for the treatment of MTB. These compounds may also act synergistically with other antimicrobial agents and thereby improve the efficiency of the therapeutic regimens or reduce the duration of treatment. The development of antibiotic-resistant strains and the major compliance issue regarding the present nearly one year treatment for the disease demands new treatment options for the number one killer of mankind by an bacterial agent.

### Example 2

### Ex Vivo Treatment of Pulmonary TB

The present example describes ax vivo studies utilizing surgical specimens obtained from patients treated by lobectomy or pulmonectomy for MDR/XDR and MTB cavitary pulmonary TB (“PTB”). This approach provides valuable information concerning active localized PTB, as compared to traditional animal model studies which following systemic (and not localized) infection with the bacterium.

In this context, it is noteworthy that conventional mammalian disease models do not afford lung cavities that are an essential component in the spread of the human form of the disease. The use of an ex vivo approach permits determination of the details of application and possible pitfalls as compared to conventional approaches in vivo for the treatment of the cavity form of the disease in clinical trials.

After obtaining a fresh surgical specimen with cavitary TB in tact, a small tissue sample from the cavity is taken through the bronchial opening and examined for M. tuberculosis. An adequate volume of 0.1 M normal saline corresponding approximately to the volume of the cavity is introduced into the cavity using a catheter fitted with an inflatable cuff. The cuff prevents and/or limits the possible leakage of the fluid from the opening leading into the cavity. This procedure maybe repeated as needed to remove any debris observed upon withdrawal of the saline into the barrel of the syringe.

After washing with saline, an equal volume of 1% of the anti-mycobacterial surfactant composition is introduced into the cavity as above and gently withdrawn from the cavity several times within approximately 10 min. If the lavage appears to contain a copious amount of cellular debris an additional volume of fresh drug solution may then be administered. After the lavage fluid appears mostly clear, the cavity is again filled with the composition for 1 hr, and the specimen is again carefully withdrawn and its content carefully observed. Upon completion of the previous step, the cavity may then be opened, and a sample of the cavitary wall tissue taken for acid fast staining of the bacteria and for culturing. The appropriate choice of cavity tissue for this study is important. Specimens with well-defined cavitary lesions of ˜2-5 cm³ are particularly contemplated as being most desirable in the ex vivo assay, which may be performed the same day of the surgery. The cavity is then assessed and grossly compared with untreated cavity as a control. These comparisons may then be used to define the requirement of time and surfactant concentration to remove all or nearly all of the bacteria from the cavity.

### Example 3

### Toxicology Studies

Toxicology studies using the anti-mycobacterial compositions were performed in mice, and the results are shown in FIG. 3. The mice, four in each group, were administered an aerosol consisting of 25 μl of saline (control) and OG or DDM (at concentrations of 0.02%, 0.2%, and 1.0%, respectively). Aerosol was introduced by the endotracheal route using a non-invasive pulmonary delivery apparatus as described by Bivas-Benita et al. (2005). Prior to introduction of the aerosol, the mice were anesthetized by the intraperitoneal injection of 200 μl of Avertin. The procedure was repeated after 24 hr and then weekly for 3 weeks. The mice were fed ad libitum, and weighed after each aerosol delivery.

One week after the regimen was completed; the lungs were removed and fixed with formalin for histological examination. Gross and microscopic changes were compared with the same in control saline-treated mice. Results from these experiments indicated that OG or DDM did not cause any tissue inflammatory response and any visible damage to the bronchial and lung structures. Histological examination of the spleens and livers also did not reveal any visible changes and no change were found in leukocyte count and in overall cytologic composition of the lavage fluid. There was also no difference in the body weight the control and experimental animals. Thus, these studies indicated that the surfactant-based intervention of the present invention does not cause any destructive or inflammatory changes in the bronchial tree or lung parenchyma, and did not cause any registered toxic effects.

### REFERENCES

The following references, to the extent that they provide exemplary procedural or other details supplementary to those set forth herein, are specifically incorporated herein by reference:

American Thoracic Society, Centers for Disease Control, and the Infectious Diseases Society of America, Joint report: “Treatment of Tuberculosis,” Morbid. Mortal. Weekly Rpt., 52(RR-11):1-77, Jun. 20, 2003.

Bivas-Benita, M. et al., “Non-invasive pulmonary aerosol delivery in mice by the endotracheal route,” Eur. J. Pharm. Biopharm., 61(3):214-8, October 2005.

Dye, C., et al., “Consensus Statement. Global burden of tuberculosis: estimated incidence, prevalence and mortality by country—WHO global surveillance and monitoring project,” JAMA, 282(7):677-86, August 1999.

Ellner, J J, “Review: The Immune response in human tuberculosis—implications for tuberculosis control,” J. Infect. Dis., 176(5):1351-1359, November 1997.

Good et al., “Hydrogen ion buffers for biological research,” Biochemistry, 5(2):467-77, February 1966.

Hwang, Park, Park, “Gastric retentive drug-delivery systems,” Crit. Rev. Ther. Drug Carrier Syst., 15(3):243-284, 1998.

Mathiowitz, Jacob, Jong, Carino, Chickering, Chaturvedi, Santos, Vijayaraghavan, Montgomery, Bassett, Morrell, “Biologically erodable microspheres as potential oral drug delivery systems,” Nature, 386(6623):410-414, 1997.

National Institutes of Health Guideline HL-95-012, “Lung specific drug delivery systems for tuberculosis treatment,”NIH Guide, 24(1):1-18, Jan. 13, 1995.

O'Callaghan et al., “Drug Delivery to the Lung,” In: “Lung Biology in Health and Disease,” Bisgaard et al., (Exec. Ed.), Vol. 162, Informa Health Care, 2002.

Russell, D G, “Mycobacterium tuberculosis: Here Today and Here Tomorrow,” Nat. Rev. Mol. Cell. Biol., 2(8):1-9, August 2001.

Saita et al., “Trehalose 6,6′-dimycolate (cord factor) of Mycobacterium tuberculosis induces corneal angiogenesis in rats,” Infect. Immun., 68(10): 5991-5997, October 2000.

Takenaga et al., “Microparticle resins as a potential nasal drug delivery system for insulin,” J. Control. Release, 52(1-2):81-87, March 1998.

World Health Organization et al., “Tuberculosis and AIDS: Statement on AIDS and Tuberculosis,” Bull. Int. Tuberc. Lung Dis., 64:88-111, 1989.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention.

More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

It will also be apparent to those skilled in the art that various changes may be made in the invention without departing from the spirit and scope thereof, and therefore, the invention encompasses embodiments in addition to those specifically disclosed in the specification.

1. A pharmaceutical composition comprising:
(a) a first surfactant selected from the group consisting of n-octanoylsucrose, n-nonanoylsucrose, n-decanoylsucrose, n-undecanoylsucrose, n-dodecanoylsucrose, n-heptyl-β-D-glucoside, n-heptyl-β-D-maltoside, n-heptyl-β-D-maltopyranoside, n-heptyl-β-D-glucopyranoside, n-heptyl-β-D-thioglucoside, n-heptyl-β-D-thiomaltoside, n-heptyl-β-D-thiomaltopyranoside, n-heptyl-β-D-thioglucopyranoside, n-octyl-β-D-glucoside, n-octyl-β-D-maltoside, n-octyl-β-D-maltopyranoside, n-octyl-β-D-glucopyranoside, n-octyl-β-D-thioglucoside, n-octyl-β-D-thiomaltoside, n-octyl-β-D-thiomaltopyranoside, n-octyl-β-D-thioglucopyranoside, n-nonyl-β-D-glucoside, n-nonyl-β-d-maltoside, n-nonyl-β-D-maltopyranoside, n-nonyl-β-D-glucopyranoside, n-nonyl-β-D-thioglucoside, n-nonyl-β- D-thiomaltoside, n-nonyl-β-D-thiomaltopyranoside, n-nonyl-β-D-thioglucopyranoside, n-decyl-β-D-glucoside, n-decyl-β-D-maltoside, n-decyl-β-D-maltopyranoside, n-decyl-β-D-glucopyranoside, n-decyl-β-thioglucoside, n-decyl-β-D-thiomaltoside, n-decyl-β-d-thiomaltopyranoside, n-decyl-β-D-thioglucopyranoside, n-undecyl-β-D-glucoside, n-undecyl-β-D-maltoside, n-undecyl-β-D-maltopyranoside, n-undecyl-β-D-glucopyranoside, n-undecyl-β-D-thioglucoside, n-undecyl-β-D-thiomaltoside, n-undecyl-β-D-thiomaltopyranoside, n-undecyl-β-D-thioglucopyranoside, n-dodecyl-β-D-glucoside, n-dodecyl-β-D-maltoside, n-dodecyl-β-D-maltopyranoside, n-dodecyl-β-D-glucopyranoside, n-dodecyl-β-D-thioglucoside, n-dodecyl-β-D-thiomaltoside, n-dodecyl-β-D-thiomaltopyranoside, n-dodecyl-β-D-thioglucopyranoside, and any combination thereof; and
(b) a first anti-Mycobacterial compound, both of which are present in the composition in an amount sufficient to treat or ameliorate a Mycobacterial infection, or one or more symptoms thereof, in a mammal to which the composition is administered.

2-45. (canceled)

46. The composition of claim 1, formulated for administration to a lung or a first surface thereof in a human that has, is suspected of having, or is at risk for developing a Mycobacterial infection.

47. The composition of claim 46, wherein the infection is caused by Mycobacterium africanum, Mycobacterium avium, Mycobacterium avium paratuberculosis, Mycobacterium avium silvaticum, Mycobacterium avium hominissuis, Mycobacterium avium colombiense, Mycobacterium botniense, Mycobacterium bovis, Mycobacterium canetti, Mycobacterium caprae, Mycobacterium haemophilum, Mycobacterium heckeshornense, Mycobacterium intracellulare, Mycobacterium kansasii, Mycobacterium leprae, Mycobacterium microti, Mycobacterium paratuberculosis, Mycobacterium pinnipdeii, Mycobacterium tuberculosis, Mycobacterium ulcerans, Mycobacterium xenopi, or any combination thereof.

48. The composition of claim 1, wherein the first anti-Mycobacterial compound is selected from the group consisting of capreomycin, cycloserine, ethambutol, ethionamide, isoniazid, kanamycin, levofloxacin, moxifloxacin, ofloxacin, pyrazinamide, rifabutin, rifampin, rifapentine, streptomycin, tobramycin, p-aminosalicylic acid, and any combination thereof

49. The composition of claim 1, further comprising at least a second distinct surfactant from the group.

50. The composition of claim 1, further comprising at least a second distinct anti-Mycobacterial compound.

51. The composition of claim 1, wherein the first surfactant is selected from the group consisting of β-D-octylglucoside, n-octyl-β-D-octylglucoside, n-dodecyl-β-D-maltoside, and any combination thereof.

52. The composition of claim 1, wherein the first surfactant is present in an amount from about 0.001 mg/ml to about 1000 mg/ml.

53. The composition of claim 11, wherein the first surfactant is present in an amount of from about 0.01 mg/ml to about 100 mg/ml.

54. The composition of claim 1, formulated for administration to the mammal by inhalation, vaporization, aerosol injection, or nebulization.

55. A method of inhibiting the growth of a population of virulent Mycobacterium cells, the method comprising contacting a suspected population of virulent Mycobacterium cells with the composition of claim 1, in an amount and for a time sufficient to at least inhibit the growth of at least a first portion of the population of virulent Mycobacterium cells.

56. The method of claim 55, wherein the population of virulent Mycobacterium cells comprises one or more Mycobacterium tuberculosis cells.

57. The method of claim 55, wherein the composition is contacted at least daily with the suspected population of virulent Mycobacterium cells for at least a period of from one day to about three months.

58. A method of killing a mycobacterial cell in a mammal, the method comprising providing to the mammal the composition of claim 1, in an amount and for a time effective to kill the mycobacterial cell in the mammal.

59. A method of treating or ameliorating one or more symptoms of a mycobacterial infection in a mammal, the method comprising administering to the mammal a therapeutically-effective amount of the composition of claim 1.

60. The method of claim 59, wherein the composition is administered to the mammal from one to about six times per day.

61. The method of claim 59, wherein the composition is administered to the mammal for a period from one day to about three months.

62. The method of claim 59, wherein the composition is administered to the mammal in a single, one-time dose.

63. A method for preventing the onset of one or more clinical symptoms of a tuberculosis infection in an individual exposed to a Mycobacterium tuberculosis bacterium, and showing a determinable count of the bacterium, but not yet experiencing the clinical symptoms of tuberculosis, the method comprising:
(a) determining the presence of one or more Mycobacterium tuberculosis cells in the sputum of an individual exposed to the bacterium;
(b) administering to the individual a therapeutically-effective amount of the composition of claim 1 for a time sufficient to prevent the onset of the one or more clinical symptoms of a tuberculosis infection in the individual; and
(c) subsequently determining the substantial absence of viable Mycobacterium tuberculosis cells in the sputum of the individual following the administering of the composition,
wherein the substantial absence of viable Mycobacterium tuberculosis cells in the sputum following administration is indicative of the effectiveness of the composition to prevent the onset of one or more clinical symptoms of a tuberculosis infection the exposed individual.

64. A method for inducing prophylaxis against tuberculosis in a mammal that may be subsequently exposed to at least a first pathogenic strain of Mycobacterium tuberculosis, the method comprising administering to the mammal a first prophylactically-effective amount of the composition of claim 1 for a time sufficient to induce prophylaxis against tuberculosis in the subsequently-exposed mammal.
